(19) United States
(12) Patent Application Publication
(10) Pub. No.: US 2007/0135756 A1
(43) Pub. Date: Jun. 14, 2007

(54) Current-Generating Pen

(76) Inventors: Philippe Kohlbrenner, Kaltacker (CH); Markus Bingesser, Wuerenlos (CH); Johannes Felber, Dietikon (CH); Kurt Heinz, Buchs (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/609,508

(22) Filed: Dec. 12, 2006

(30) Foreign Application Priority Data
Dec. 13, 2005 (DE) 10 2005 059 508.1

(51) Int. Cl. A61N 1/30

(52) U.S. Cl. 604/21

(57) ABSTRACT

An injection device including an energy producing device for producing electrical energy from non-electrical energy and an energy storing device for storing the electrical energy produced, encompassing a method of producing and storing electrical energy in an injection device wherein electrical energy is produced from non-electrical energy and stored in the energy storing device.

### CROSS-REFERENCE TO RELATED APPLICATIONS

This application claims priority to German Application No. 10 2005 059 508.1, filed on Dec. 13, 2005, the content of which is incorporated herein by reference in its entirety.

### BACKGROUND

The present invention relates to devices for administering, injecting, delivering, infusing or dispensing substances, and to methods of making and using such devices. More particularly, the present invention relates to an injection device and a method of producing and storing electrical energy in the injection device. More particularly, it relates to an injection device and a method of producing and storing electrical energy, whereby the electrical energy can be generated in the injection device from non-electrical energy, for example from mechanical energy or light energy, and in some embodiments, stored in the injection device.

Some embodiments of injection devices have electrical components or systems. Such devices need electrical energy to enable or power the electronic system, which, for example, may include such devices as an electronic counting system, a display, sensors, etc.

Batteries are used in conventional injection devices but their service life is limited and largely depends on external factors, which prevents or impairs use of the injection devices under specific conditions, for example in a tropical climate.

### SUMMARY

An object of the present invention is to provide an injection device comprising a feature and/or features enabling the “on-board” production and storage of electrical energy. Another object is to provide a method of producing and storing electrical energy, which ensures a reliable and long-lived supply of electrical energy.

In one embodiment, the present invention comprises an injection device comprising an energy producing device for producing electrical energy from non-electrical energy.

In one embodiment, the present invention comprises an injection device comprising an energy producing device for producing electrical energy from non-electrical energy and an energy storing device for storing the electrical energy produced.

In one embodiment, the present invention comprises a method of producing electrical energy in an injection device wherein the electrical energy is produced from non-electrical energy.

In one embodiment, the present invention comprises a method of producing and storing electrical energy in an injection device wherein the electrical energy is produced from non-electrical energy and stored in the energy storing device.

In one embodiment, an injection device in accordance with the invention has an energy-generating device, by which electrical energy can be generated or produced from non-electrical energy associated with or produced by the injection device. In some embodiments, for the purpose of the present invention, the injection device also has an energy storing device in which the electrical energy generated in the energy generating device can be permanently or temporarily stored. In some preferred embodiments, the energy generating device may be designed so that it produces or generates electrical energy from mechanical energy or converts mechanical energy into electrical energy.

In some embodiments, the energy generating device comprises a magnet, such as a permanent magnet or an electromagnet which may be of a cylindrical design, comprising a coil with at least one turn. The coil and the magnet may be encased, for example in a casing of soft iron or other suitable material. The diameter of the coil or the circumference of a turn of the coil is larger than the base surface of the permanent magnet or than the diameter of the permanent magnet, so that the magnet can be moved relative to the coil and/or be moved by the coil. If the magnet is moved with respect to the coil or in a direction toward the coil, a voltage is induced in the coil so that an electric current is able to flow through the coil.

In some embodiments, the coil may be connected to a spring, which may be connected to a re-set or return button or feature associated with the injection device. When the re-set is operated, the spring may be released so that the permanent magnet can be moved relative to the coil or into the coil, thereby causing or enabling a voltage, current or energy to be induced in the coil. The permanent magnet or the magnet may also be mounted so that movements of the injection device or components thereof, such as driving or injecting movements, guiding movements or movements of the injection device in space, cause the magnet to move relative to the coil, thereby enabling a voltage or a current to be induced due to the movement of the magnet relative to the coil.

In some embodiments, the energy generating device may also or additionally have a stator and a rotor and the rotor may be connected to a rotating button, dose metering feature or a setting mechanism of the injection device, in some embodiments via a gear mechanism. This being the case, the rotor, which may be of a cylindrical shape, may be rotatably mounted inside the stator or the stator may be cylindrical in shape, in which case the rotor may be mounted so that it can rotate about the stator. In some embodiments, the stator has at least one coil with at least one or more turns and the rotor is provided in the form of a magnet. In some embodiments, the rotor may be a permanent magnet, for example a cylindrical permanent magnet or an electromagnet, which may have one or more turns, by which an electric current can be conducted so that a magnetic field can be generated around the rotor. Due to the rotation of the rotor or the rotating motion of the rotor, the magnetic field at the stator varies virtually constantly, so that a voltage or a current or energy can be induced in the stator.

The rotor may be moved or rotated in a number of ways in various embodiments. For example, the rotor may be connected to the rotating button of the injection device so that when the rotating button is turned, for example when setting a dose, the rotor rotates accordingly. The rotor may also be mounted so that movements of the injection device induce or cause a rotation of the rotor.

In some embodiments of the present invention, the energy generating device may comprise a rotatably mounted flywheel, which can be set in motion or rotated by the movements of the injection device. This flywheel may be connected to a generator, either directly or via a gear mechanism for example, which is able to produce electrical energy from the rotating motion of the flywheel. The electrical energy may then be used to supply an electronic system or display of the injection device directly or it may be stored, for example in an energy storing device.

In some embodiments, the energy producing device may comprise a piezoelectric generator, which may comprise a ceramic piezoelectric element, a piezoelectric film or a piezoelectric film sensor. When vibrations are induced in the piezoelectric element, film or film sensor due to mechanical movements acting on a tongue of the piezoelectric element, for example, a voltage, in particular a sinusoidal alternating voltage, may be produced by the piezoelectric generator. This alternating voltage may be converted into a direct voltage by means of a transformer and a low-pass filter, which may be used to operate the electronic system or display of the injection device, or which can initially be stored in an energy storage device.

In some embodiments, the energy producing device may also or additionally be designed so that it is able to convert optical energy, in particular light energy, into electrical energy or can produce electrical energy from optical energy. To this end, the energy producing device may comprise at least one solar cell, at least one photo-diode or alternatively two, three, four or five photo-diodes, which may be connected in series, in which case the photo-diode or solar cell is able to convert incident optical energy or incident light into electrical energy. The solar cell may be operated in conjunction with the energy producing devices described above or in combination with a battery so that the voltage of the solar cell can be monitored by a control circuit or processor, and/or the injection device may be switched to battery mode if the voltage drops. In some embodiments, display may comprise a light-emitting diode to indicate that the injection device is operating in battery mode.

In some embodiments, the energy storing device in which the electrical energy produced or generated can be stored is provided in the form of a capacitor, such as a super capacitor, double layer capacitor or gold capacitor, for example, or as a battery, for example in the form of a rechargeable battery or accumulator. If current is needed to operate the electronic system, the display or another function or feature of the injection device, the energy storing device may output the stored energy, making current available.

In accordance with the present invention, in some embodiments of the method of producing and storing electrical energy in an injection device electrical energy is produced from or generated by non-electrical energy in an energy producing device of the injection device. The electrical energy produced can then be stored in an energy storing device of the injection device or may be used to operate the electronic system of the injection device. In some preferred embodiments, electrical energy may be drawn from mechanical or optical energy, e.g. light energy.

In some preferred embodiments, a longitudinal movement or a linear movement of a permanent magnet or an electromagnet through a coil or along a coil may generate electrical energy or an electric voltage or an electric current in the coil, which can be forwarded to an energy storing device of the injection device. The magnet may be connected to a rotating button or feature or a setting button of the injection device which is manipulated, e.g., depressed, to dispense the product, thereby permitting a longitudinal movement along the longitudinal axis of the injection device so that the magnet is able to effect a longitudinal movement and move into the coil. The magnet could also be moved by movements of the injection device and moved into the coil so that an electrical energy can be generated. The magnet may also be connected to a re-setting device or feature of an injection device which can be operated when a dose-setting button is pressed.

In some embodiments, when the re-setting device is operated, a dose-setting button moves in the direction opposite the dispensing direction of the operating mechanism of the injection device, along the longitudinal axis of the injection device. If the magnet is connected to the dose-setting button, for example, a movement of the dose-setting button also causes a movement of the magnet and voltage is induced in the coil due to the relative movement with respect to the coil.

In some embodiments, an energy producing device in accordance with the present invention may utilize a turning or rotating movement to produce electrical energy. In particular, a magnetic rotor may effect a rotating movement relative to a stator with at least one coil to induce electrical energy in the stator. The rotor may be rotated or moved in a number of ways. For example, the rotor may be moved relative to the stator by movements of the injection device which are transmitted to the rotor. The rotor may also be connected to a rotating button or a dose setting mechanism of the injection device. For example, when a user turns the dose setting button to set a desired dose, the rotor also turns and induces a voltage in the stator due to the changing magnetic field, which can be stored in an energy storing device for operating the electronic system of the injection device.

In some embodiments, energy stored in a spring may be used to produce electrical energy. For example, the injection device may have a biased spring which may have an operating mechanism on the external surface of the injection device. When the operating mechanism is activated, the spring is released so that spring energy can drive or move a rotor or a magnet, e.g., in a longitudinal movement. Mechanical movements of the injection device or mechanical movements of parts of the injection device, such as a longitudinal movement of a dose setting button or a rotating movement of a dose setting button, may be transmitted to a piezoelectric sensor or a piezoelectric element, such as a ceramic piezoelectric element, a piezoelectric film or a piezoelectric film sensor to a piezoelectric element, e.g., a tongue. Due to the mechanical movements, the piezoelectric sensor generates an electric voltage or electrical energy. If periodic movements or repeated movements are transmitted to or act on the piezoelectric component it may generate sinusoidal alternating voltages, which can be converted into direct voltage by a transformer and a low-pass filter. The direct voltage may be stored in the energy storing device, for example.

In some preferred embodiments, electrical energy may also or additionally be produced from optical energy, such as light. For example, if at least one solar cell or a photo-diode is provided on the injection device, electrical energy can be generated from light energy by the solar cell or photo-diode and transmitted to and stored in an energy storing device. A control circuit or suitable processor may be used to monitor and indicate when sufficient electrical energy can no longer be generated from the light energy, or when it is no longer possible to store sufficient electrical energy. In some embodiments, such a control circuit or processing feature may redirect the energy demand to a different energy producing cycle or to a battery, for example.

### BRIEF DESCRIPTION OF THE DRAWINGS

FIG. 1a illustrates an embodiment of an energy producing device of the present invention, comprising a bar magnet 10, a coil 11 and a casing 12 with a comb system 13;

FIG. 1b illustrates a second embodiment of an energy drawing device of the present invention with a bar magnet 10, a coil 11 and a casing 12 with a comb system 13;

FIG. 2 shows the rear part of the injection device of another embodiment of the present invention;

FIG. 3 shows a circuit of an energy drawing device of another embodiment of the present invention comprising a battery 31 and solar cells 30; and

FIG. 4 shows another embodiment of an energy drawing device of the present invention with solar cells 40.

### DETAILED DESCRIPTION

With regard to fastening, mounting, attaching or connecting components of the present invention to form the invention, unless specifically described as otherwise, conventional mechanical fasteners and methods may be used. Other appropriate fastening or attachment methods include friction fitting, adhesives, welding. soldering, etc. The electrical system, electrical features or electrical components may comprise suitable electrical components, e.g., circuitry, wires, chips, boards, microprocessors, power sources, photo-voltaic devices, power storage devices, communication devices (e.g., transmitters, receivers, etc.), inputs, outputs, sensors, displays, control components, etc. Generally, unless otherwise indicated, the materials for making the device of the present invention and/or its components may be selected from appropriate materials such as metals, metallic alloys, ceramics, plastics, etc.

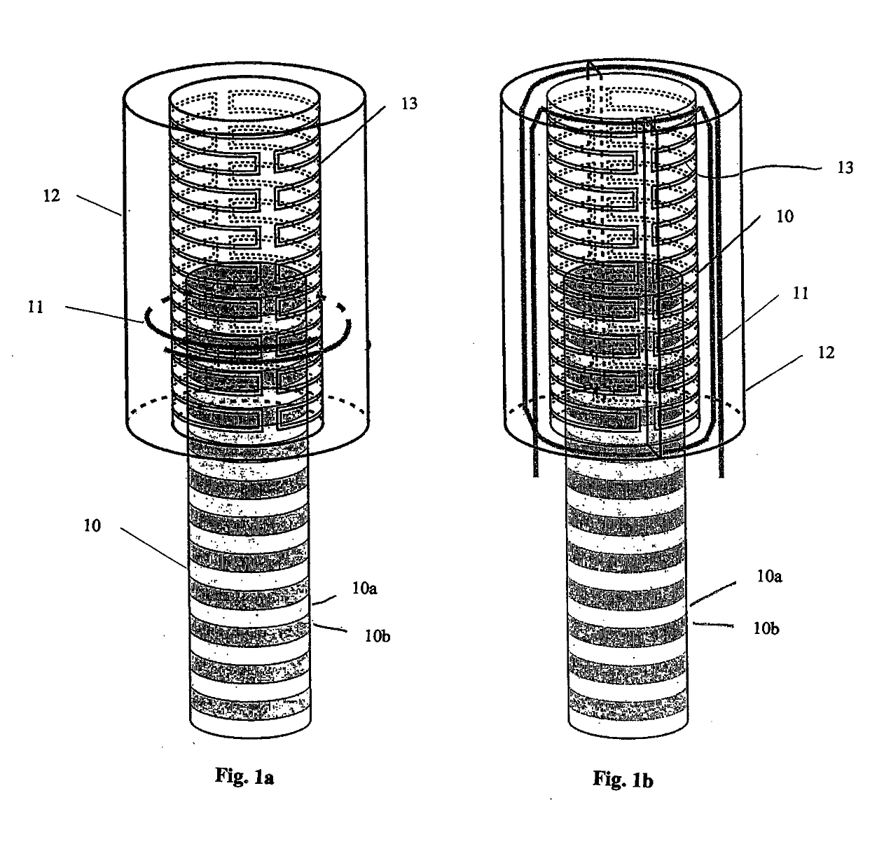

FIG. 1a illustrates a first embodiment of an energy producing device integrated in an injection device in accordance with the present invention. The energy producing feature or device, comprises a bar magnet or magnetic bar 10 and a coil 11 with at least one turn, the turns extending horizontally with respect to the base surface of the magnet 10. The bar magnet is a permanent magnet and therefore permanently generates a magnetic field. In particular, the magnet or bar magnet 10 has a plurality of poles 10a, 10b, between which a plurality of magnetic fields may be generated. The radius or diameter of the coil 11 is larger than the radius or diameter of the base surface of the bar magnet 10 so that the bar magnet 10 can be moved or pushed into or fed through the coil 11. The coil 11 is also surrounded by a soft iron casing 12 with a soft iron comb system 13. When the bar magnet 10 moves into the coil 11, for example due to a movement, e.g. shaking, of injection device, an electric voltage, an electric current or electrical energy is induced in the coil 11, which can be used to operate the injection device or temporarily stored in a storage device. In some embodiments, the bar magnet 10 may alternatively be moved due to the fact that the bar magnet 10 is connected to a push-button or dose setting button of the injection device. When the push-button is moved, the bar magnet 10 also moves into the coil 11 or out of the coil 11. The bar magnet 10 is also able to move along the coil 11, into the coil 11 or out of the coil 11 due to the fact that the bar magnet 10 is mounted to move due to movements of the injection device.

In some embodiments, the magnet 10 may be connected to a re-set button of the injection device via a biased spring. When the re-set button or the return mechanism is operated, the dose setting mechanism or dose setting button is moved in the direction opposite the dispensing direction of the injectable product, so that the magnet 10 can also be moved along the longitudinal axis of the injection device and moves relative to the coil 11, thereby enabling a voltage or a current to be induced in the coil 11. If the bar magnet has a plurality of poles 10a, 10b, such as north and south poles disposed in an alternating arrangement, a magnetic field can be generated between each north and each south pole so that a plurality of magnetic fields can be generated along the bar magnet. When the bar magnet 10 is pushed into the coil 11, each of these magnetic fields is able to exert a force on the charges or particles in the coil or each turn of the coil or separate the charges or particles in the coil or induce a voltage or a current in the coil 11 or in the turns of the coil 11.

FIG. 1b illustrates the energy producing device shown in FIG. 1a, but with a coil 11 having turns running vertically with respect to the base surface of the bar magnet 10 instead of a horizontal coil 11.

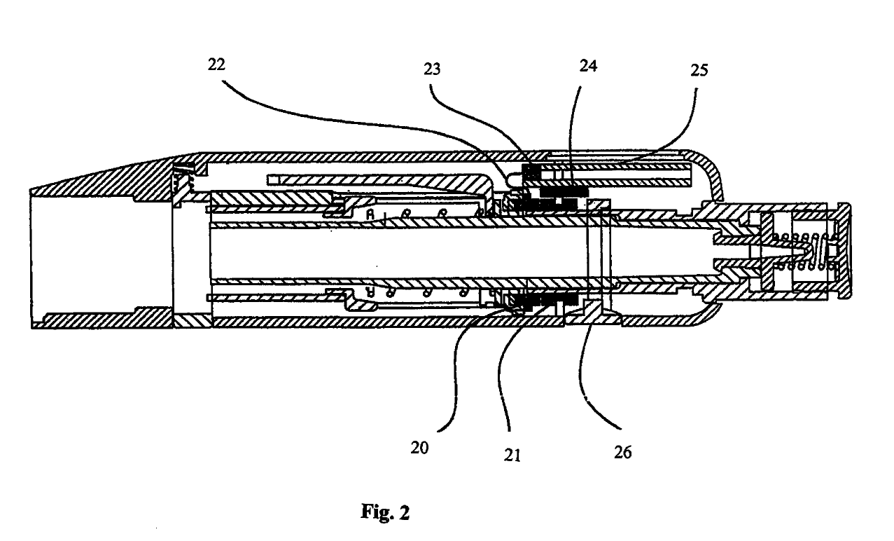

FIG. 2 illustrates the rear part of an injection device in accordance with the present invention with a rotor 21 in the form of a magnetic ring, a stationary stator 20, an electrical connection 22 between the stator 20 and an energy storage 23, reed contacts 24, an LCD display 25 and a re-set switching ring 26. When the rotor 21 is moved or rotated in a circumferential direction relative to the stator 20 due to movement of the injection device, for example, a voltage or energy can be generated in the stator 20, which can be stored in the energy storage 23 via the electrical connection 22. The LCD display 25 may be supplied with current by the stored energy.

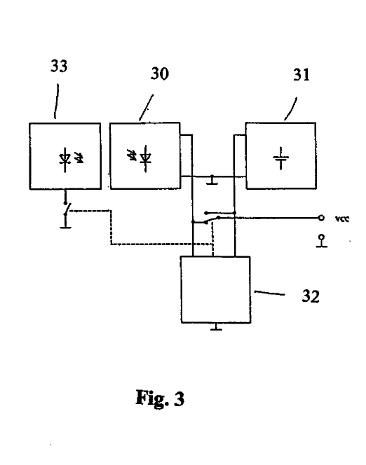

FIG. 3 illustrates another embodiment of an energy drawing device in accordance with the present invention comprising solar cells 30, a battery 31, a control circuit 32 and a light-emitting diode (LED) 33. By means of the solar cells 30, incident light energy can be converted into electrical energy and stored in an energy storing device. The control circuit 32 is able to detect whether there is still enough light to enable sufficient energy to be stored in the energy storing device. When sufficient light or energy is no longer available, the supply is switched to the battery 31 so that the electronic system and the display are supplied by the battery 31. During battery mode, the LED 33 is activated, providing a display to indicate that there is not enough light or light energy to produce electrical energy.

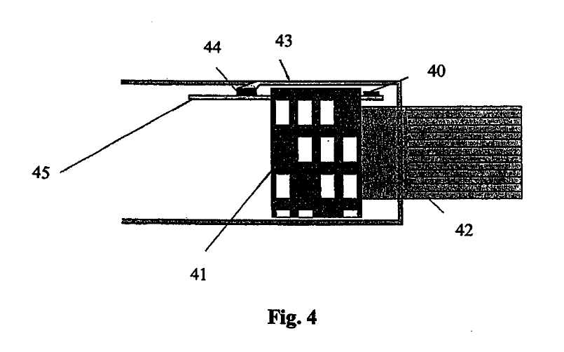

FIG. 4 illustrates another embodiment of an energy producing device in accordance with the present invention comprising photo-diodes 40, disposed on a circuit board 45, a light guide 43 in the form of a window for incident light, a light-emitting diode 44, a dose setting button 42 of the injection device and a dial 41 enabling the selected or set dose or metered dose to be detected. When the dose setting button 42 is changed to set or select a dose, the dial 41 moves along the circumference of the injection device. Depending on the selected setting, a different window or orifices are disposed above the photo-diodes 40, or other windows of the dial 41 open, and enable light to hit the photo-diodes 40. When light hits the photo-diodes 40, electrical energy is generated from the light by means of the photo-diodes 40, which can be stored in an energy storing device associated with or carried in. The produced energy may also be used, in whole or in part, directly, i.e., without being stored.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection device comprising an energy producing device for producing electrical energy from non-electrical energy.

2. An injection device comprising an energy producing device for producing electrical energy from non-electrical energy and an energy storing device for storing the electrical energy.

3. The injection device as claimed in claim 2, wherein the energy producing device produces electrical energy from mechanical energy.

4. The injection device as claimed in claim 2, wherein the energy producing device comprises a permanent magnet and a coil with at least one turn.

5. The injection device as claimed in claim 4, wherein the permanent magnet is connected to a spring connected to a re-setting mechanism of the injection device, and wherein when the re-setting mechanism is operated the spring is released and moves the permanent magnet relative to the coil.

6. The injection device as claimed in claim 4, wherein the coil is circular, the permanent magnet is cylindrical and the circumference of the coil is larger than the circumference of a base surface of the permanent magnet.

7. The injection device as claimed in claim 6, wherein the permanent magnet is connected to a spring connected to a re-setting mechanism of the injection device, wherein when the re-setting mechanism is operated the spring is released and the permanent magnet moves relative to the coil.

8. The injection device as claimed in claim 2, wherein the energy producing device comprises a stator and a rotor connected to one of a rotating structure or dose setting structure of the injection device.

9. The injection device as claimed in claim 8, wherein the rotor is connected to one of the rotating structure or dose setting structure of the injection device via a gear mechanism.

10. The injection device as claimed in claim 8, wherein the rotor is one of rotatably mounted inside the stator or mounted so that it can rotate about the stator.

11. The injection device as claimed in claim 8, wherein the stator comprises a coil with at least one turn and the rotor comprises a magnet.

12. The injection device as claimed in claim 11, wherein the magnet is one of a permanent magnet or an electromagnet.

13. The injection device as claimed in claim 2, wherein the energy producing device comprises a rotatably mounted flywheel and a generator and produces electrical energy from the rotating motion of the flywheel.

14. The injection device as claimed in claim 2, wherein the energy producing device comprises a piezoelectric generator comprising one of a ceramic piezoelectric element, a piezoelectric film or a piezoelectric film sensor, and produces electrical energy from mechanical movements acting on one of the ceramic piezoelectric element, piezoelectric film or piezoelectric film sensor.

15. The injection device as claimed in claim 2, wherein the energy producing device produces electrical energy from optical energy.

16. The injection device as claimed in claim 15, wherein the energy producing device comprises one of at least one solar cell or photo-diode which produces electrical energy from optical energy.

17. The injection device as claimed claim 2, wherein the energy storing device is a capacitor.

18. The injection device as claimed claim 17, wherein the capacitor is one of a super capacitor, a double-layer capacitor or a gold capacitor.

19. The injection device as claimed in claim 2, wherein the energy storing device is a battery.

20. The injection device as claimed in claim 19, wherein the battery is rechargeable.

21. A method of producing and storing electrical energy in an injection device, wherein electrical energy is produced from non-electrical energy by an energy producing device of the injection device and the electrical energy is stored in an energy storing device.

22. The method as claimed in claim 21, wherein the non-electrical energy is at least one of mechanical and optical energy.

23. The method as claimed in claim 21, wherein a longitudinal movement of a permanent magnet through a coil is converted into electrical energy.

24. The method as claimed in claim 21, wherein a turning or rotating movement of a magnetic rotor inside or about a stator with at least one coil is converted into electrical energy.

25. The method as claimed in claim 21, wherein energy stored in a spring is converted into electrical energy.

26. The method as claimed in claim 21, wherein mechanical movement of one of a ceramic piezoelectric element, a piezoelectric film or a piezoelectric film sensor is converted into electrical energy.

27. The method as claimed in claim 21, wherein optical energy hitting one of a solar cell or photo-diode associated with the injection device is converted into electrical energy.

28. The method as claimed in claim 21, wherein mechanical energy produced by a movement of the injection device is converted into electrical energy.

29. The method as claimed in claim 28, wherein the mechanical energy produced by the movement of the injection device is stored in a spring and converted into electrical energy when the spring is released or relaxed.
